(19) United States
(12) Patent Application Publication
(10) Pub. No.: US 2025/0252631 A1
(43) Pub. Date: Aug. 7, 2025

(54) IMAGE PROCESSING APPARATUS, IMAGE PROCESSING METHOD, AND NON-TRANSITORY COMPUTER READABLE MEDIUM

(71) Applicant: CANON KABUSHIKI KAISHA, Tokyo (JP)

(72) Inventors: Yuta NARUKIYO, Kanagawa (JP); Ryo ISHIKAWA, Kanagawa (JP); Kiyohide SATOH, Kanagawa (JP)

(21) Appl. No.: 19/042,031

(22) Filed: Jan. 31, 2025

(30) Foreign Application Priority Data
Feb. 5, 2024 (JP) 2024-015884

(51) Int. Cl. G06T 11/60; G06T 7/33; G16H 30/40

(52) U.S. Cl. CPC G06T 11/60; G06T 7/337; G16H 30/40; G06T 2207/20221

(57) ABSTRACT

An image processing apparatus according to the present disclosure includes: an image acquisition unit configured to acquire a first image which has an abnormal region in a first region, and a second image which is different from the first image and includes at least the first region, by imaging at least one subject; a region information acquisition unit configured to acquire region information on the abnormal region in the first image; an registration information acquisition unit configured to acquire registration information related to registration of the first image and the second image; and a synthetic image generation unit configured to combine the first image and the second image based on the region information and the registration information, and generate a synthetic image which has a region corresponding to the abnormal region in the first region.

### BACKGROUND OF THE INVENTION

### Field of the Invention

The present invention relates to an image processing apparatus, an image processing method, and a non-transitory computer readable medium.

### Description of the Related Art

In a case of performing image recognition and class classification using machine learning, it is known that learning various patterns of data as teacher data contribute to the improvement of recognition accuracy and classification accuracy.

For example, Zhang, H., et al. “mixup: Beyond empirical risk minimization. ICLR 2018.” arXiv preprint arXiv: 1710.09412 (2017). discloses a method of improving detection accuracy by combining images in-between different classes (e.g. dogs and cats) at a predetermined ratio and generating an image of a pattern interpolating a decision boundary between the classes. Further, Japanese Patent Application Publication No. 2020-018705 discloses a method of generating pseudo images of different case patterns from a single image based on the compositional brightness distribution of pulmonary nodules, so as to alleviate the insufficiency of patterns of the data.

However, in the case of Zhang, H., et al. “mixup: Beyond empirical risk minimization. ICLR 2018.” arXiv preprint arXiv: 1710.09412 (2017). the positional relationship of the images to be combined is not considered, and therefore in a case of generating a pseudo image of a lesion, for example, simply superimposing images may combine regions outside of the target organ, and a desirable pseudo image cannot be generated. Further, in Japanese Patent Application Publication No. 2020-018705, a method of combining images before and after generating the lesion is not disclosed.

### SUMMARY OF THE INVENTION

With the foregoing in view, it is an object of the technique of the present disclosure to alleviate the insufficiency of patterns of the image data by generating pseudo images based on the registration information between images of a subject.

According to some embodiments, an image processing apparatus includes: one or more processors; and a memory storing a program which, when executed by the one or more processors, causes the image processing apparatus to execute: image acquisition processing to acquire a first image which has an abnormal region in a first region, and a second image which is different from the first image and includes at least the first region, by imaging at least one subject; region information acquisition processing to acquire region information on the abnormal region in the first image; registration information acquisition processing to acquire registration information related to registration of the first image and the second image; and synthetic image generation processing to combine the first image and the second image based on the region information and the registration information, and generate a synthetic image which has a region corresponding to the abnormal region in the first region.

In addition, according to some embodiments, an image processing method includes: a step of acquiring a first image which has an abnormal region in a first region, and a second image which is different from the first image and includes at least the first region, by imaging at least one subject; a step of acquiring region information on the abnormal region in the first image; a step of acquiring registration information related to registration of the first image and the second image; and a step of combining the first image and the second image based on the region information and the registration information, and generating a synthetic image which has a region corresponding to the abnormal region in the first region.

Further features of the present invention will become apparent from the following description of exemplary embodiments with reference to the attached drawings.

### BRIEF DESCRIPTION OF THE DRAWINGS

FIG. 1 is a block diagram depicting a general configuration of an image processing apparatus according to Embodiment 1.

FIG. 2 is a flow chart of processing executed by the image processing apparatus according to Embodiment 1.

FIG. 3 is a schematic diagram for describing determination of a combining ratio according to Embodiment 1.

FIG. 4 is a schematic diagram depicting an overview of registration and combining processing according to Embodiment 1.

FIG. 5 is a block diagram depicting a general configuration of an image processing apparatus according to Embodiment 2.

FIG. 6 is a flow chart of processing executed by the image processing apparatus according to Embodiment 2.

### DESCRIPTION OF THE EMBODIMENTS

Embodiments of an image processing apparatus disclosed in the present description will now be described with reference to the drawings. A same or similar composing element, member or processing step in each drawing is denoted with the same reference sign, and redundant description may be omitted unless necessary. In each drawing, a part of composing elements, members or processing steps may be omitted.

In the following, the present invention will be described using CT image data captured by an X-ray computer tomographic imaging (CT) apparatus as an example of medical image data. Embodiments of the present invention are not limited to the following embodiments, and are also applicable to images captured by a nuclear magnetic resonance imaging (MRI) apparatus, a position tomographic imaging (PET) apparatus, or an ultrasonic diagnostic apparatus.

### Embodiment 1

An image processing apparatus according to Embodiment 1 executes processing to generate a synthetic image (pseudo image), including a simulated lesion, from an image of a subject, as synthetic image generation processing. In Embodiment 1, the image processing apparatus acquires a first image (current image) which was captured at a certain timing and includes an abnormal region, and a second image (prior image) which was captured in the past before the timing of capturing the current image and does not include an abnormal region of the subject. Then the image processing apparatus executes registration of the first image and the second image to match pancreas regions (first regions) depicted in the images. The lesion in Embodiment 1 is a pancreatic cancer, and the abnormal region is a pancreatic cancer region. By combining pancreatic cancer regions using the image generated by registering the pancreas region in the prior image with the pancreas region in the current image, and the current image, the image processing apparatus generates a pseudo image indicating an early stage pancreatic cancer. The target of the image composition is not limited to the pancreas region, but may be various organ regions of the subject. Further, the lesion is not limited to the pancreatic cancer, but may be lesions of various organs, such as liver and kidneys.

Generally, a five year survival rate from pancreatic cancer is clearly lower than cancers of other organs, and in many cases, when pancreatic cancer has grown too large to be removed at point of detection. Hence early detection of pancreatic cancer is critical, but this is difficult since ultrasound from the body surface does not reach the pancreas very well, which is located at the center region of the body, and early pancreatic cancer has few subjective symptoms, which causes a delay in detailed examination.

For the early stage detection of pancreatic cancer by image diagnosis, support of image processing using machine learning models, such as convolutional neural network (CNN) and random forest, is effective. In this case, it is critical to collect case data of early stage pancreatic cancer. However, as mentioned above, the early stage detection of pancreatic cancer is difficult, so collecting case data of early stage pancreatic cancer is difficult, except in cases of accidental detection.

Therefore in Embodiment 1, a synthetic image reproducing a simulated early stage pancreatic cancer is generated using a contrast-enhanced CT image (current image) of a pancreatic cancer case captured at a certain timing, and a contrast-enhanced CT image (prior image) captured before the above timing when the subject was healthy. Here both the current image and the prior image are contrast-enhanced CT images, but are not necessarily contrast-enhanced CT images, and may be non-contrast CT images. The positions of the two images, of which image capturing timings are different, deviate for various reasons, such as changes in the body, progress of lesion, breathing, and image capturing methods, and a desired synthetic image cannot be acquired simply by combining the same pixels of the two images. Therefore, in Embodiment 1, registration processing is performed to register the pancreas region in the prior image with the pancreas region in the current image. The same effect can be implemented even if the current image is registered with the prior image. In Embodiment 1, the combining processing is performed mainly targeting the region where the pancreatic cancer exists, hence the information on the region where the pancreatic cancer exists (pancreatic cancer region information) in the current image is acquired. Then the synthetic image that reproduces the simulated early stage pancreatic cancer is generated from the current image, the registered prior image, and the pancreatic cancer region information.

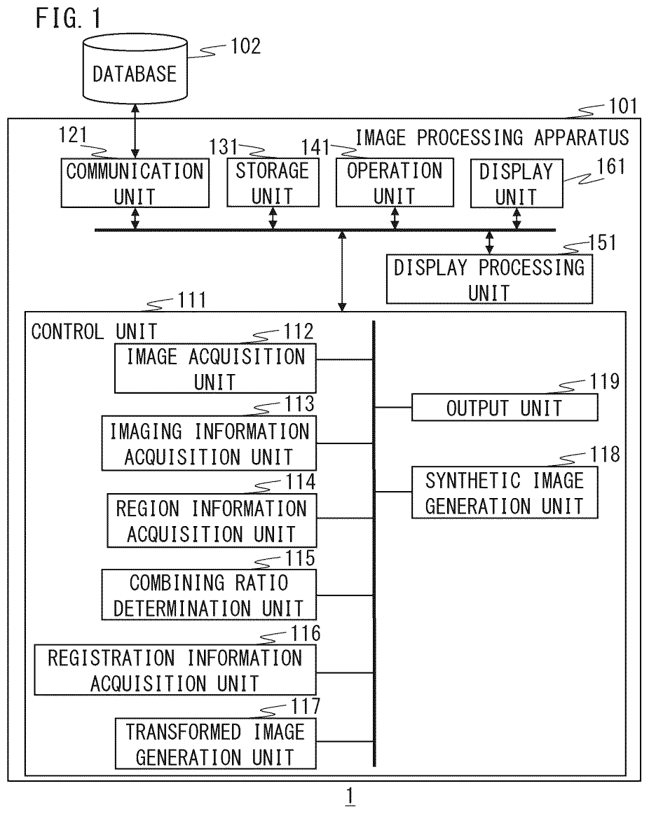

A configuration and processing of the image processing apparatus according to Embodiment 1 will be described with reference to FIG. 1. FIG. 1 is a block diagram depicting a general configuration example of an image processing system 1, which includes the image processing apparatus 101 according to Embodiment 1. As indicated in FIG. 1, the image processing apparatus 101 includes a control unit 111, a communication unit 121, a storage unit 131, an operation unit 141, a display processing unit 151, and a display unit 161, according to Embodiment 1. The image processing apparatus 101 is connected to an external database 102 of the image processing apparatus 101 via the communication unit 121.

The control unit 111 is constituted of a central processing unit (CPU) and a dedicated or general purpose processor. The control unit 111 may also be constituted of a graphic processing unit (GPU) or a field-programmable gate array (FPGA). Further, the control unit 111 may be constituted of an application specific integrated circuit (ASIC) or the like. The control unit 111 implements the image processing method according to the technique of the present disclosure by controlling each component in the image processing apparatus 101, and executing the processing described below.

The image acquisition unit 112 acquires a current image and a prior image from the storage unit 131. The current image is, for example, an image of a target object of the subject (patient) acquired by a CT diagnostic apparatus, and in Embodiment 1, a contrast-enhanced CT image of a pancreatic parenchymal phase of an abdominal region, when about 40 to 50 seconds have elapsed from ejection of contrast medium, is used. However, the contrast time phase is not limited to the above, and the effect of the present invention can be expected in other time phases, such as a portal phase, an arterial phase, an equilibrium phase, and a late phase. Further, a non-contrast CT image may be used without performing contrast imaging. The prior image is an image of the target object of the subject captured at a timing in the past before the current image. The contrast time phase and the image capturing conditions of the prior image are preferably the same as the current image, but may be different. The image acquisition unit 112 may acquire the current image directly from the CT diagnostic apparatus, and in this case, the image processing apparatus 101 may be installed in the CT diagnostic apparatus that provides part of the functions of the CT diagnostic apparatus.

An imaging information acquisition unit 113 acquires such imaging information as time phase information (contrast imaging information), imaging date and time, and reconstruction function, from the image acquired by the image acquisition unit 112. The imaging information may be stored in the storage unit 131 as a separate file, and in this case, the imaging information acquisition unit 113 acquires the imaging information from the database 102 or the storage unit 131. In a case where information on the progress degree (abnormality progress degree), such as a size of a pancreatic cancer and progress level, is attached to the imaging information, the imaging information acquisition unit 113 also acquires the attached progress degree information. Further, the imaging information acquisition unit 113 may acquire the above imaging information by user input via the operation unit 141.

The region information acquisition unit 114 acquires a label image of a pancreatic cancer from the storage unit 131, as pancreatic cancer region information to indicate a position of the pancreatic cancer in the current image. Further, the region information acquisition unit 114 acquires label images of a pancreas region from the storage unit 131, as respective pancreas region information of the current image and the prior image. For example, the label image of the pancreatic cancer is a binary label image where the pancreatic cancer region is 1 and the other regions are 0. The label image of the pancreas region is also a binary label image where the pancreas region is 1 and the other regions are 0. The pancreatic cancer region information is not limited to the label image (binary image) of the pancreatic cancer, but may be any form of information if the information indicates the position of the pancreatic cancer in the current image. For example, the region information acquisition unit 114 may acquire a likelihood image which holds a value of a likely pancreatic cancer region for each pixel as the pancreatic cancer region information, or may acquire the coordinates of the representative position of the pancreatic cancer. The pancreas region information may also be any form if the information indicates the pancreas region. The region information acquisition unit 114 may acquire the pancreatic cancer region information and the pancreas region information by user input via the operation unit 141.

A combining ratio determination unit 115 is a ratio information acquisition unit to acquire combining ratio information on the ratio of combining the first image and the second image in the synthetic image. Specifically, the combining ratio determination unit 115 acquires the information on the progress degree of the lesion and the lesion region from the imaging information acquisition unit 113 and the region information acquisition unit 114 respectively, and determines the combining ratio to combine the current image and the prior image. The combining ratio determination unit 115 may determine the combining ratio by user input via the operation unit 141.

A registration information acquisition unit 116 registers the pancreas region in the current image and the pancreas region in the prior image using the label image of the pancreas region acquired by the region information acquisition unit 114, and acquires the information on displacement between the images as the result of this processing. The information on displacement between the images here may be information indicating translational shift, rotation, enlargement, reduction, or the like, or may be information indicating a local deformation, or may be information indicating a combination of these displacements.

A transformed image generation unit 117 executes the coordinate transformation on the prior image based on the registration information acquired by the registration information acquisition unit 116, and generates a transformed image in which the pancreas region approximately matches with the current image. The generated transformed image may be stored in the storage unit 131.

A synthetic image generation unit 118 generates a synthetic image by combining the current image and the prior image, based on the transformed image generated by the transformed image generation unit 117, the pancreatic cancer region information acquired by the region information acquisition unit 114, and the combining ratio determined by the combining ratio determination unit 115.

An output unit 119 stores the synthetic image generated by the synthetic image generation unit 118 to the database 102 or the storage unit 131. Instead of or in addition to this, the output unit 119 may output the synthetic image to the display processing unit 151, and display the synthetic image on the display unit 161.

A communication unit 121 can be connected to an external device or network, and implements communication using predetermined communication means. The communication unit 121 may be constituted of a wireless device, such as Wi-Fi and Bluetooth (registered trademark). Further, the communication unit 121 may be constituted of a cable device, such as a cable local area network (LAN) and a universal serial bus (USB). The image processing apparatus 101 communicates with an external database using predetermined communication means via the communication unit 121, and acquires at least one image and various data, such as region information.

The storage unit 131 is constituted of at least one medium storing data, such as a hard disk drive (HDD) and a random access memory (RAM). The storage unit 131 stores various data and various calculation results to be processed by the image processing apparatus 101. The storage unit 131 may be configured by a main storage constituted of a volatile memory which temporarily stores read data or the like, and an auxiliary storage which stores data long term.

The operation unit 141 is constituted of input devices, such as a keyboard, a mouse, a touch panel, and a remote controller, to input instructions from the user to various devices. The display processing unit 151 processes images and various calculation results received from the control unit 111 in a form with which the display unit 161 can display, and transfers the processing result to the display unit 161. The display unit 161 is constituted of an output device, such as a display, and displays display data, such as calculation results and various images processed by the display processing unit 151, to the user.

The database 102 stores various images to be processed by the image processing apparatus 101. The database 102 may also store processing results and output images in the image processing apparatus 101. The database 102 may be on a network, or may be physically connected with the image processing apparatus 101.

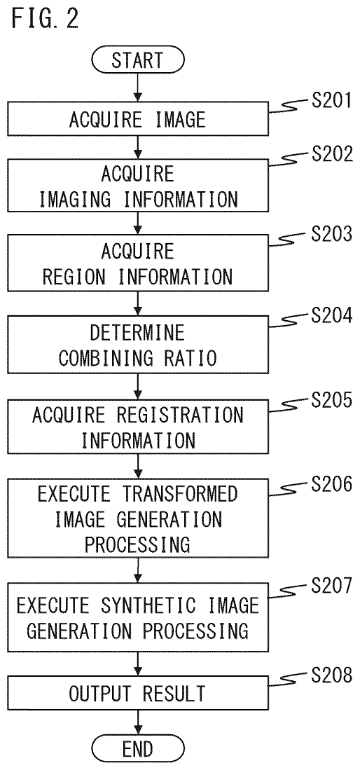

Now an example of the processing executed by the image processing apparatus 101 according to Embodiment 1 will be described in detail with reference to the flow chart in FIG. 2. In the following description, it is assumed that the current image and the prior image acquired by imaging the subject have been stored in the storage unit 131.

(Step S201: Acquire Image) In step S201, the image acquisition unit 112 acquires the current image and the prior image, which the user specified via the operation unit 141, from the storage unit 131. The images may be acquired by other methods. For example, the image acquisition unit 112 may communicate with the database 102 and acquire the current image and the prior image that satisfy predetermined conditions, from the database 102. Here the display processing unit 151 may display the input images, acquired by the image acquisition unit 112, on the display unit 161. The images acquired by the image acquisition unit 112 here are a current image in which the pancreatic cancer is depicted, and a prior image capturing the subject in the past where the pancreatic cancer is not depicted. The image acquisition unit 112 may acquire a plurality of prior images, and in this case, the processing steps after step S202 are executed for each pair of a plurality of pairs of the current image and the plurality of prior images. In Embodiment 1, the current image and the prior image are three-dimensional contrast images captured by the CT diagnostic apparatus, but the current image and the prior image may be other modalities, non-contrast images or two-dimensional images. Further, the current image and the prior image may also be a multi-time phase image group captured at plurality of contrast imaging timings.

(Step S202: Acquire Imaging Information) In step S202, the imaging information acquisition unit 113 acquires the imaging information, such as the time phase information (contrast imaging information), imaging date and time, reconstruction function, and resolution, from the image acquired in step S201. If information on the progress degree, such as the size and progress level of the pancreatic cancer, is attached to the imaging information, the imaging information acquisition unit 113 may acquire this information as the progress degree information. The progress degree information is, for example, the size of the cancerous tumor, the degree of metastasis in other organs, and a number of metastases in lymph nodes. The current image and the prior image are preferably images captured in the same contrast imaging time phase (e.g. pancreatic parenchymal phase), but may be images captured in different contrast image time phases (e.g. current image is in pancreatic parenchymal phase and prior image is in portal phase). In the case where the contrast imaging time phase of the current image is different from that of the prior image, the imaging information acquisition unit 113 may perform brightness correction to correct the difference of the average brightness of each pancreas region generated due to the difference of the degree of staining of the contrast medium, for the current image and/or the prior image. Further, the reconstruction function and resolution (number of pixels) may be different between the current image and the prior image. If the resolution (number of pixels) of the current image is different from that of the past time, the imaging information acquisition unit 113 may additionally perform processing to transform the current image and/or the prior image so as to be the same resolution (number of pixels).

Furthermore, in a case where a multi-time phase image group is acquired in step S201 as input images, the imaging information acquisition unit 113 may perform time phase selection processing to select a pair of the current image and the prior image of which similarity of the imaging conditions is highest. For example, the imaging information acquisition unit 113 acquires the contrast imaging time or imaging time information from the auxiliary information of the image included in the digital imaging and communication in medicine (DICOM) information. Then the imaging information acquisition unit 113 can estimate the time phase closest to the pancreatic parenchymal phase, and select the current image and the prior image of which time phases are close to the estimated time phase. Further, in a case where the current image is an image other than the pancreatic parenchymal phase and the prior image is in the multi-time phase group, the imaging information acquisition unit 113 may selectively acquire a prior image of which imaging condition is most similar to the current image. The imaging conditions of the current image and the prior image may be estimated using a known method, such as deep learning.

(Step S203: Acquire Region Information) In step S203, the region information acquisition unit 114 acquires information on the pancreatic cancer region indicating the position of the pancreatic cancer in the current image. Specifically, the region information acquisition unit 114 acquires the label information of the pancreatic cancer in the current image from the storage unit 131. The label image of the pancreatic cancer indicating the position of the pancreatic cancer is, for example, a binary label image where the pancreatic cancer region is 1 and the other regions are 0. The label image of the pancreatic cancer may be a multi-value image with Gaussian distribution, of which peak position is the center of the pancreatic cancer. Here the region information acquisition unit 114 may change the spread of the distribution based on the distance between the center of the pancreatic cancer and the contour of the pancreas region. The information on the pancreatic cancer region may be a circumscribed rectangle surrounding the pancreatic cancer, which may be a label image where the internal region of the circumscribed rectangle is 1 and the other regions are 0, or may be the coordinate values of the apex positions of the circumscribed rectangle may be used instead. Further, the region information acquisition unit 114 may acquire information on the pancreatic cancer region by user input, and in this case, the current image may be outputted to the display processing unit 151 and displayed on the display unit 161, and input of the region specified by the user may be received via the operation unit 141. In the case of receiving user input, the region information acquisition unit 114 may acquire the region of the pancreatic cancer in pixel units based on the operation with a mouse or the like, or may acquire the region in such a form as coordinate values of the circumscribed rectangle.

Besides the information on the pancreatic cancer region, the region information acquisition unit 114 acquires information on the pancreas region in each image of the current image and the prior image. The region information acquisition unit 114 may acquire the information on the pancreas region from the storage unit 131, or may be acquired by the user input, just like the case of the information on the pancreatic cancer region described above. Further, the region information acquisition unit 114 may acquire the information on the pancreas region in the form of the label image, or may acquire the information in the form of the circumscribed rectangle surrounding the pancreas region, just like the case of the information on the pancreatic cancer region described above. Furthermore, the region information acquisition unit 114 may automatically acquire the information on the pancreas region by image processing of the current image and the prior image (e.g. segmentation by a known method).

If the information on the pancreatic cancer region and the information on the pancreas region acquired above are inconsistent, the region information acquisition unit 114 may additionally execute processing to correct the acquired information. For example, in the case of acquiring the pancreatic cancer region based on the specification by the user, this pancreatic cancer region may extend outside of the pancreas region in the current image. In this case, the region information acquisition unit 114 corrects the pancreatic cancer region specified by the user, based on the information on the pancreas region in the current image.

(Step S204: Determine Combining Ratio) In step S204, the combining ratio determination unit 115 acquires information on the progress degree of the lesion and the lesion region in the current image and the prior image from the imaging information acquisition unit 113 and the region information acquisition unit 114, and determines the combining ratio to combine the current image and the prior image. Here the combining ratio determination unit 115 may determine the combining ratio by user instruction via the operation unit 141, or the combining ratio determination unit 115 may determine the combining ratio based on the imaging information acquired in step S202. Specifically, the combining ratio determination unit 115 can determine the combining ratio based on the progress degree of the pancreatic cancer in the imaging information.

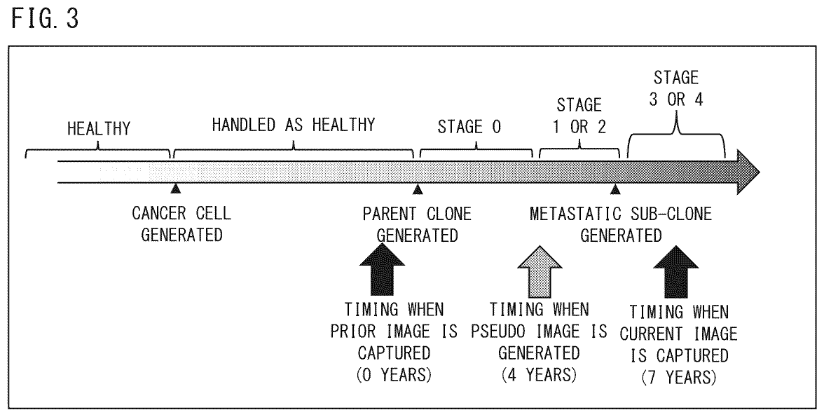

In Embodiment 1, an image of a pancreatic cancer stage 1 is generated from the pancreatic cancer image of the pancreatic cancer stage 3, for example. FIG. 3 indicates the relationship between the imaging timing and the progress of the pancreatic cancer in Embodiment 1. Generally, an average number of years in the progress of pancreatic cancer is statistically known. In Embodiment 1, it is assumed that it takes 11 years from the generation of cancer cells of the pancreatic cancer to the generation of parent clones; 6 years from the generation of parent clones to the generation of metastatic sub-clones; and 3 years from the generation of metastatic sub-clones to death. In Embodiment 1, images captured up to the timing of the generation of parent clones is treated as images of a healthy case (that is, cancer cells are not generated (regarded as 0 years)).

Here it is assumed that the current image was captured when 1 year elapsed since the generation of metastatic sub-clones, and 7 years elapsed from the imaging timing of the prior image to the imaging timing of the current image. It is also assumed that stage 1 indicates the timing when 4 years elapsed since the generation of parent clones. In this case, the timing at the start of stage 1 is a point determined by internally dividing the time from the imaging timing of the prior image (0 years) to the imaging timing of the current image (7 years) by the ratio 4:3. Therefore by combining the prior image and the current image by the ratio 3:4, a desired image simulating the state of stage 1 can be generated. The above mentioned statistical average number of years may be freely changed by the user. The progress degree in the pseudo image to be generated may be arbitrarily determined using a random number of the like.

The method of determining the combining ratio is not limited to the above mentioned method based on the difference between the image timings, but the combining ratio may be determined based on the stage estimated by the size of the pancreatic cancer in the image, for example. In this case, the size of the pancreatic cancer can be acquired using the pancreatic cancer region information acquired in Step S203, and if the length of one side of the pancreatic cancer is 30 mm, for example, the cancer is regarded as stage 3. The pancreatic cancer stage may also be determined by user input. For example, it is assumed that the length of one side of the pancreatic cancer in the current image is 30 mm; the length of one side of the pancreatic cancer in the prior image is 0 mm; and the length of one side of the pancreatic cancer in the pseudo image to be generated is 10 mm. In this case, the timing of the pancreatic cancer in the pseudo image to be generated is a timing determined by internally dividing the elapsed time from the imaging timing of the prior image to the imaging timing of the current image by the ratio 1:2. Therefore by combining the prior image and the current image by the ratio 2:1, a desired pseudo image can be generated.

Furthermore, the combining ratio determination unit 115 may determine the combining ratio based on the size of the pancreatic cancer, regardless of the stage of the pancreatic cancer. The combining ratio determination unit 115 calculates the size of the pancreatic cancer using the pancreatic cancer region information acquired in step S203, in the same manner as the determination method based on the stage of the pancreatic cancer. For example, it is assumed that the length of one side of the pancreatic cancer in the prior image is 0 mm, the length of one side of the pancreatic cancer in the pseudo image to be generated is 10 mm, and the length of one side of the pancreatic cancer in the current image is 30 mm. In this case, the timing of the pancreatic cancer in the pseudo image to be generated can be regarded as a timing determined by internally dividing the elapsed time from the imaging timing of the prior image to the imaging timing of the current image by the ratio 1:2, based on the ratio of the length of the one side of the pancreatic cancer in each image. Therefore, by combining the prior image and the current image by the ratio 2:1, a desired pseudo image can be generated. The length of the one side of the pancreatic cancer in the pseudo image to be generated may be determined by user input via the operation unit 141.

Furthermore, the combining ratio determination unit 115 may also determine the combining ratio based not only on the size of the pancreatic cancer, but also on image features, such as pixel values of the lesion and the contrast of the lesion with the peripheral region. In this case, the combining ratio determination unit 115 acquires pixel values (CT values) of the pancreatic cancer region from the pancreatic cancer region information acquired in step S203, and acquires pixel values (CT values) of the pancreas region (pancreatic parenchymal region) from the pancreas region information, excluding the pancreatic cancer region, in the same manner. Based on the pixel values of each of these regions, the combining ratio determination unit 115 calculates the contrast ratio of the brightness between the pancreatic cancer region and the pancreatic parenchymal region.

For example, the contrast ratio is set to a value determined by dividing an average pixel value of the pancreatic cancer region by an average pixel value of the pancreatic parenchymal region. In the pancreatic cancer in an early stage, the degree of staining of the contrast medium is normally similar to that of the pancreatic parenchymal phase, where the above contrast ratio is small and hard to visually recognize. Therefore, the combining ratio determination unit 115 can determine the combining ratio based on the contrast ratio. For example, if the contrast ratio of the pancreatic cancer region in the current image is 2, and the contrast ratio of the pancreatic cancer region in the pseudo image to be generated is 1.5, then the combining ratio determination unit 115 sets the combining ratio of the prior image and the current image to 1:1. Thereby a desired pseudo image can be generated. Further, the contrast ratio of the pancreatic cancer region in the pseudo image to be generated may be determined by user input via the operation unit 141.

Therefore the combining ratio determination unit 115 can determine the combining ratio using a method not dependent on the difference of imaging timings. In this case, the image processing apparatus 101 need not execute the imaging information acquisition processing in step S202. The combining ratio in this step need not always be dynamically determined, and, for example, the combining ratio of the current image and the prior image may be set to a predetermined fixed ratio, such as 1:1.

(Step S205: Acquire Registration Information) In step S205, the registration information acquisition unit 116 acquires the registration information to approximately match the position and form of the pancreas region in the prior image with the position and form of the pancreas region in the current image, based on the pancreas region information acquired in step S203. The registration information in Embodiment 1 is information on the displacement for each pixel in the prior image to be shifted to a pixel in the current image, and if the prior image is a three-dimensional image, the registration information is information having motion vectors in the X, Y and Z directions for each pixel. For specific processing to acquire the registration information, such known methods as template matching, elastic net, and large deformation diffeomorphic metric mapping (LDDMM) can be used. Further, feature points may be detected in the pancreas region in the current image and the pancreas region in the prior image using a known method, and the motion vectors indicating the coordinate transformation to approximately match the corresponding feature points may be acquired as the registration information.

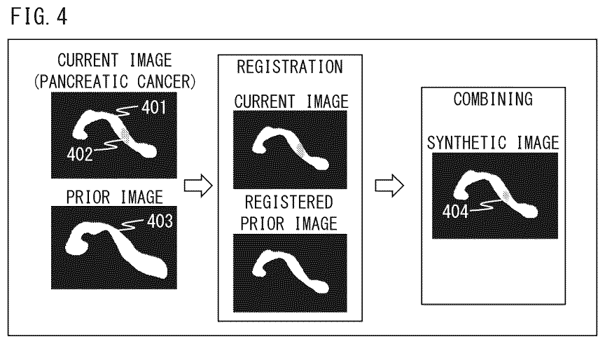

(Step S206: Execute Transformed Image Generation Processing) In step S206, the transformed image generation unit 117 generates a transformed image by performing the registration processing to register the prior image with the current image, based on the registration information acquired in step S205. Specifically, for the transformed image, the transformed image generation unit 117 generates an image by transforming the coordinates of the prior image to match with the current image. In Embodiment 1, as illustrated in FIG. 4, the transformed image generated by executing the coordinate transformation processing on the prior image is outputted. However, in Embodiment 1, the transformation processing is not limited to this transformation processing exemplified in FIG. 4, and the transformed image may be generated by executing the coordinate transform processing on the current image to match with the prior image.

In FIG. 4, a pancreas region 401 and a pancreatic cancer region 402 are depicted in the current image, and a pancreas region 403 is depicted in the prior image. The transformed image generation unit 117 executes the transformation processing to make the position and form of the pancreas region 403 in the prior image close to the position and form of the pancreas region 401 in the current image, based on the registration information acquired in step S205. In the transformation processing, it is not always necessary to use the pancreas region information acquired in step S203, and the transformation processing can also be executed using the registration information acquired in step S205. If the pancreatic cancer region depicted in the current image is located outside of the pancreas region in the transformed image in this transformation processing, correction processing may be executed using the pancreas region information, so that the pancreatic cancer region in the current image is located within the pancreas region in the transformed image. Further, the transformed image generation unit 117 may perform the registration processing in step S205 under the condition where the pancreatic cancer region in the current image is located in the pancreas region in the transformed image.

The above registration processing may be executed based on the pancreas region information of the current image and the prior image acquired in step S203. Further, the above registration processing may register the current image and the prior image themselves. In the case of the registration of these images, the pancreatic cancer regions may not be registered appropriately because the presence of the pancreatic cancer region is different between the current image and the prior image. To best handle this situation, it is preferable to execute processing to correct the result of registering the pancreatic cancer regions based on the result of registering the peripheral areas of the pancreatic cancer regions. In the case of executing the registration of images by a method not using the pancreas region information, such as the case of registering the current image and the prior image themselves, the image processing apparatus 101 need not acquire the pancreas region information in step S203.

(Step S207: Execute Composite Image Generation Processing) In step S207, the synthetic image generation unit 118 generates a synthetic image by combining the current image and the transformed image based on the result of the transformed image generation unit 117, the pancreatic cancer label acquired in step S203, and the combining ratio determined in step S204. In Embodiment 1, the pancreatic cancer region in the current image and a corresponding region in the transformed image are combined. Here the corresponding region in the transformed image is a region in the transformed image corresponding to the pancreatic cancer region in the current image. In the generation of the synthetic image, the pixel values of the pancreatic cancer region in the current image and the pixel values of the corresponding region in the transformed image are weighted and combined based on the combining ratio determined in step S204. For example, if the combining ratio is determined as 3:4 in step S204, the pixel values of the synthetic image are determined by adding the pixel values of the corresponding region in the prior image multiplied by 3/7 and the pixel values of the pancreatic cancer region in the current image multiplied by 4/7. In this case, the pixels of the region, other than the pancreatic cancer region in the current image, are not combined with the transformed image, and the pixel values in the current image are regarded as the pixel values in the synthetic image. As a result, as illustrated in FIG. 4, the pancreatic cancer region 402 in the current image is generated as a shadow portion 404, simulating the pancreatic cancer in the early stage in the synthetic image by the combining processing. In this processing step, the current image and the transformed image are directly combined, but the transformed image is an image generated by transforming coordinates of the prior image in the processing in step S206, hence this processing substantially corresponds to the processing combining the current image and the prior image.

In some cases of combining the pancreatic cancer region in the current image and the corresponding region in the transformed image, an artifact may be generated at the boundary of the region to be combined (pancreatic cancer region) and the other region due to the combining processing. The influence of an artifact on the synthetic image can be reduced if the transformed image generation unit 117 performs the combining processing by smoothly changing the combining ratios spatially in the peripheral region of the boundary using Gaussian distribution, for example. The transformed image generation unit 117 may also generate a synthetic image reducing the size of the pancreatic cancer region by adjusting the position of the boundary and degree of smoothness.

(Step S208: Output Result) In step S208, the output unit 119 stores the synthetic image of the early stage pancreatic cancer generated in step S207 in the storage unit 131. Further, in the case of using the generated synthetic image for a task of extracting the pancreatic cancer region of a machine learning model (segmentation), the output unit 119 may store the pancreatic cancer region information of the current image in the storage unit 131 as the correct pancreatic cancer region information (correct data) of the generated synthetic image. Furthermore, in the case where the combining ratio was determined using the pancreatic cancer stage in step S204, the output unit 119 may store the pancreatic cancer stage after generation of the synthetic image in the storage unit 131 as auxiliary information of the synthetic image.

As described above, according to the processing of the image processing apparatus 101 according to Embodiment 1, the current image and the prior image are combined based on the registration information between the images, whereby a synthetic image of simulated early stage pancreatic cancer can be generated.

Modifications of the above mentioned Embodiment 1 will now be described. In the following description, a composing element or processing the same as the composing element or processing of the image processing apparatus 101 described above will be denoted with the same reference sign, and detailed description thereof will be omitted.

### Modification 1-1

In Embodiment 1, the case of generating the synthetic image by combining the pancreatic cancer region in the current image and the corresponding region in the transformed image, as the processing in step S207, was described as an example, but the target regions of the combining processing are not limited to this. For example, the pancreas regions, other than the pancreatic cancer region, such as a pancreatic ducts, are also deformed by the generation of the pancreatic cancer, hence a synthetic image including the changes in these regions may be generated. Specifically, the synthetic image generation unit 118 generates the synthetic image targeting not only the pancreatic cancer region but the entire pancreas region including the pancreatic cancer region. Here the synthetic image generation unit 118 need not use the pancreatic cancer region information to combine the images, and may execute the registration processing using only the pancreas region information. In this case, the processing to acquire the pancreatic cancer information in step S203 may be omitted. However, if the pancreatic cancer region information is not used, the texture of other organs, such as pancreatic ducts, may be mixed and combined in the pancreatic cancer region, hence it is preferable that the registration processing includes registration processing for other organs and tumors (e.g. pancreatic ducts, cysts). The other organs and tumors (e.g. pancreatic ducts, cysts) may be detected by a known method, such as deep learning, or may be specified by the user via the operation unit 141.

The method of generating the synthetic image is not limited to the method of combining the transformed image, which is generated by transforming the coordinates of the prior image, and the current image, but may be a method of generating an image indicating an intermediate state between the current image and the prior image using such a known technique as morphing. In this case, it is not necessary to transform the pancreas region in the prior image in accordance with the pancreas region in the current image, but may be transformed into an intermediate form of the forms of the pancreas regions in these images. The form of the pancreas region in the synthetic image need not be an intermediate form of the forms of the pancreatic regions in these images, but may be, for example, a form closer to the form of the pancreas region in the current image, or a form closer to the form of the pancreas region in the prior image. Further, based on the combining ratio determined in step S204, the form of the pancreas region in the current image or the pancreas region in the prior image may be determined that the form of the pancreas region in the synthetic image is closer. In this case, the shape of the pancreas region in the synthetic image may be determined by allocating moving amount each feature point of the prior image and the current image for an amount based on the combining ratio, for example. Furthermore, a number of synthetic images to be generated need not always be one, instead a plurality of synthetic images having different forms of the pancreas region may be generated.

As described above, according to Modification 1-1, the image processing apparatus 101 can generate a pseudo image where not only the state of the pancreatic cancer region, but the form of the pancreas and the state of other organs and tumors are also highly correlated with the state of the early stage pancreatic cancer.

### Modification 1-2

In Embodiment 1, it is assumed that the image processing apparatus 101 uses the current image and the prior image of the same subject. However, the prior image may be an image that does not include a pancreatic cancer of another subject (that is, an image before generating parent clones). In this case, as the prior image, the image acquisition unit 112 may acquire an image capturing another subject having subject information similar to the subject information on the subject of the current image from the database 102 or the storage unit 131. The subject information here includes, for example, the patient information (e.g. gender, age, weight, size of pancreas), imaging apparatus, reconstruction function, and imaging facility. Further, if the imaging information acquisition unit 113 also uses the imaging information acquired from the database 102 or the storage unit 131, the image acquisition unit 112 can specify another subject that is highly similar to the subject of the current image. Furthermore, the image acquisition unit 112 may select a prior image of which volume of the pancreas region is at least the same level as in the current image, or may randomly select an image from a plurality of candidates of the prior image.

As described above, according to Modification 1-2, the image processing apparatus 101 processes images acquired from at least one subject. Then in a case where the image before the pancreatic cancer was generated does not exist or cannot be acquired as a prior image of the subject of the current image, the image processing apparatus 101 can generate a synthetic image using the image of another subject based on this current image, as the prior image.

### Modification 1-3

In Embodiment 1, in steps S205 and S206, registration processing is executed to approximately match the position and form of the pancreas region in the prior image and the position and form of the pancreas region in the current image, then the transformed image is generated. However, in this registration processing, transforming the pancreas region may be omitted. For example, this registration processing may be performed by a parallel shift and/or enlargement and reduction.

For example, a case of executing the registration processing using a circumscribed rectangle of the pancreas region will be described. In step S205, the registration information acquisition unit 116 specifies the form of the circumscribed rectangles of the respect pancreas regions in the current image and the prior image acquired by the region information acquisition unit 114. Here the information on the circumscribed rectangles may be acquired based on the user input via the operation unit 141. Then the registration information acquisition unit 116 calculates the moving amount of the center of the circumscribed rectangle of the prior image, in order to match the center of the circumscribed rectangle of the past with the center of the circumscribed rectangle in the current image. Then the registration information acquisition unit 116 calculates the enlarging amount or the reducing amount of the circumscribed rectangle of the prior image, to match the size of the circumscribed rectangle of a prior image with the size of the circumscribed rectangle of the current image. Here the registration information acquisition unit 116 may calculate the enlarging amount or the reducing amount in each X, Y and Z axis direction in the coordinate system of the prior image. Thereby the registration information acquisition unit 116 acquires the calculated information as the registration information.

Then in step S206, the transformed image generation unit 117 generates the transformed image by transforming the prior image using the registration information acquired in the above processing. In the case where the pancreatic cancer region in the current image is located outside of the pancreas region in the transformed image, the form and/or position of the pancreas region in the prior image is adjusted, so that the pancreas region in the transformed image is located within the pancreas region in the current image.

As described above, according to the image processing apparatus 101 of Modification 1-3, the pancreas regions in the current image and the prior image can be registered by an easy method.

### Modification 1-4

In Embodiment 1, the image processing apparatus 101 combines one prior image with the current image. However, a plurality of prior images may be used to generate one synthetic image. In this case, an image generated by averaging the pixel values of each image of the plurality of prior image is acquired, and the acquired image is combined with the current image. For example, in step S205, for each of the acquired prior images, the registration information acquisition unit 116 acquires the registration information between the prior image and the current image. Then in step S206, the transformed image generation unit 117 generates a plurality of transformed images by transforming each of the prior images based on the registration information acquired in step S205. Then the transformed image generation unit 117 generates the image by averaging the pixel values of the plurality of transformed images. Then in step S207, the synthetic image generation unit 118 generates a synthetic image by combining the image generated in step S206 and the current image.

As described above, according to the image processing apparatus 101 of Modification 1-4, the synthetic image can be generated using a plurality of prior images before the pancreatic cancer was generated, hence a synthetic image, which is strongly resistant to changes during the imaging of the lesions depicted in an individual prior image, can be generated.

The method of generating the synthetic image using a plurality of prior images according to Modification 1-4 is not limited to the above method, but may be a method of selecting one prior image appropriate for combining with the current image, out of a plurality of prior images. For example, out of the imaging information of the DICOM information, an image, of which time phase and the imaging apparatus are closest to the imaging information of the current image, may be selected. In this case, the synthetic image generation unit 118 registers the selected prior image with the current image, and generates the synthetic image thereby. Since images of which imaging conditions are very similar are combined, the generation of a synthetic image having higher quality can be expected.

Further, the synthetic image generation unit 118 may create a plurality of pairs of the current image and a prior image using the current image and a plurality of prior images, so as to generate synthetic images for a number of the prior images. Thereby the image processing apparatus 101 can generate more synthetic images.

### Modification 1-5

In Embodiment 1, the synthetic image is generated in step S207 after generating the transformed image in step S206. However, the synthetic image may be generated omitting the generation of the transformed image. For example, the synthetic image generation unit 118 may specify the pixels of the current image and the prior image corresponding to each pixel in the synthetic image, based on the registration information acquired in step S205, and for each specified pixel, a value combining each pixel value of the current image and the prior image is regarded as each pixel value of the synthetic image.

Then the transformed image generation unit 117 of the image processing apparatus 101 becomes unnecessary, and the registration information acquisition unit 116 sends the acquired registration information to the synthetic image generation unit 118, whereby the synthetic image can be generated more efficiently, omitting the above mentioned processing to generate the transformed image.

### Modification 1-6

In Embodiment 1, the image processing apparatus 101 generates one synthetic image from one set of the current image and the prior image. However, a plurality of synthetic images may be generated from one set of the current image and the prior image. For example, the combining ratio determination unit 115 determines a plurality of combining ratios to combine the current image and the prior image. Then the synthetic image generation unit 118 generates a synthetic image based on each combining ratio which was determined, whereby a plurality of synthetic image are generated. For example, the combining ratio determination unit 115 calculates a plurality of combining ratios by varying the combining ratio acquired in step S204. Specifically, the combining ratio determination unit 115 varies the combining ratio acquired in step S204 using a random value or the like, so as to determine a plurality of combining ratios.

According to the image processing apparatus 101 of Modification 1-6, many synthetic images can be generated from one set of the current image and the prior image.

### Modification 1-7

In Embodiment 1, the registration information acquisition unit 116 calculates the registration information in step S205. However, the registration information acquisition unit 116 may acquire a predetermined registration information from the outside. For example, the registration information acquisition unit 116 acquires the registration information from the storage unit 131. Thereby processing to calculate the registration information can be omitted, and a more efficient processing can be expected.

Further, in step S206, the transformed image generation unit 117 may acquire the transformed image from the outside, without performing the processing to generate the transformed image. In this case, the registration information need not be acquired in step S205.

### Embodiment 2

An image processing apparatus according to Embodiment 2 will be described next. In the following description, a composing element or processing the same as a composing element or processing of the image processing apparatus 101 according to Embodiment 1 will be denoted with the same reference sign, and detailed description thereof will be omitted.

The image processing apparatus according to Embodiment 2 combines a current image and a prior image to generate a synthetic image simulating an early stage pancreatic cancer, then assigns a teacher label to this image, and learns a machine learning model which extracts (infers) a pancreatic cancer from the image. The image processing apparatus according to Embodiment 1 generates a synthetic image simulating an early stage pancreatic cancer, whereas the image processing apparatus according to Embodiment 2 learns the machine learning model using the synthetic image.

The image processing apparatus according to Embodiment 2 further includes: a teacher label assignment unit which assigns a teacher label to a synthetic image simulating an early stage pancreatic cancer; an inference image acquisition unit which acquires an inference image; and an inference unit which extracts a pancreatic cancer from the inference image. The image processing apparatus according to Embodiment 2 also includes a learning unit which learns a machine learning model used by the inference unit.

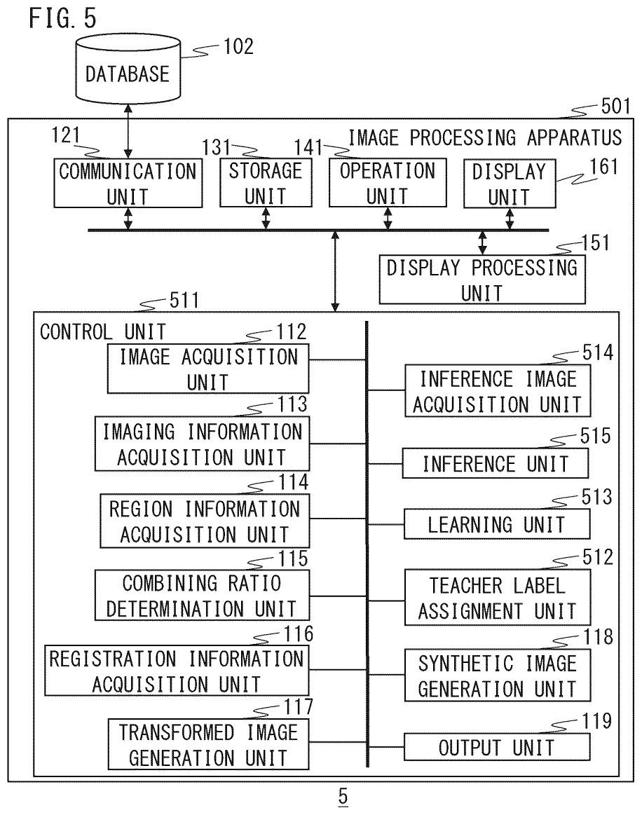

A configuration of the image processing apparatus according to Embodiment 2 will now be described with reference to FIG. 5. FIG. 5 is a block diagram depicting a general configuration of an image processing system 5 which includes an image processing apparatus 501 according to Embodiment 2. As indicated in FIG. 5, in addition to each component of the control unit 111 of the image processing apparatus 101, a control unit 511 of the image processing apparatus 501 includes a teacher label assignment unit 512 which assigns a teacher label to a synthetic image, and a learning unit 513 which performs learning of a machine learning model using the synthetic image and the teacher label. The control unit 511 also includes an inference image acquisition unit 514 which acquires an image for inference, and an inference unit 515 which performs inference using the learned machine learning model.

The teacher label assignment unit 512 acquires the combining ratio determined by the combining ratio determination unit 115 and the synthetic image generated by the synthetic image generation unit 118, and assigns a teacher label to the synthetic image. The learning unit 513 acquires a synthetic image from the synthetic image generation unit 118, acquires a teacher label from the teacher label assignment unit 512, and learns a machine learning model, which extracts a pancreatic cancer region from the image, using the acquired synthetic image and the teacher label. The learning unit 513 may also acquire a current image and a prior image from the image acquisition unit 112, and use the acquired images for learning the machine learning model. Further, the learning unit 513 may acquire images of cases, which are not used for the generation of the synthetic image and which the teacher label has been assigned to, from the database 102 or the storage unit 131, and use the acquired images for learning the machine learning model. The inference image acquisition unit 514 acquires an inference image, to be inputted to the learned machine learning model, from the storage unit 131. The image used for inference may be a synthetic image used for the learning unit 513, or other images for learning. The inference unit 515 inputs the inference image, acquired by the inference image acquisition unit 514, to the machine learning model learned by the learning unit 513, and acquires an output image, in which a pancreatic cancer region is extracted, as the inference result.

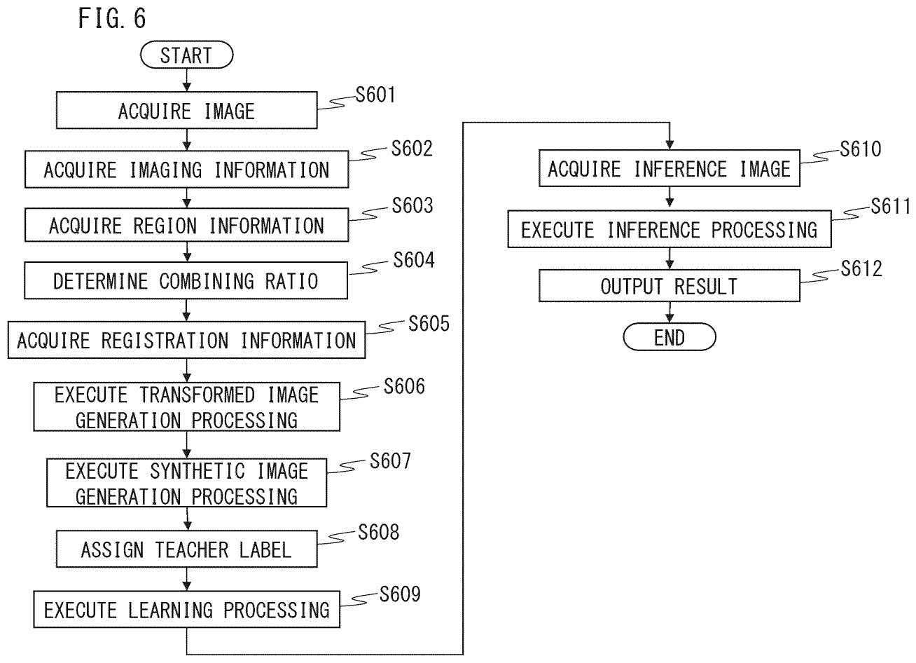

Now an example of the processing executed by the image processing apparatus 501 in FIG. 5 will be described in detail with reference to the flow chart in FIG. 6. The processing in steps S601 to S607 are the same as the processing in steps S201 to S207 in the flow chart of Embodiment 1 respectively. In the following description, it is assumed that the input image of the machine learning model has been stored in the storage unit 131 of the image processing apparatus 501.

(Step S608: Assign Teacher Label) After the image processing apparatus 501 executes the processing in step S601 to S607, the teacher label assignment unit 512 assigns in step S608 a teacher label to an abnormal region in the generated synthetic image. The teacher label here is a label image indicating a region of a pancreatic cancer (pancreatic cancer region). Here the prior image is registered to the current image, but the current image may be registered to the prior image to generate the synthetic image, just like the case of the modifications of Embodiment 1. In this case, the teacher label assignment unit 512 assigns a label image of the pancreatic cancer region, generated by registering the current image to the prior image (transforming coordinates) using the registration information, to the synthetic image as the teacher label. The teacher label assignment unit 512 also assigns a teacher label to a synthetic image in the same manner in the case of generating an intermediate image between the current image and the prior image as the synthetic image. The label image of the pancreatic cancer region used in Embodiment 2 is a binary image, which has the same resolution (number of pixels) as the synthetic image, where the pixel values of the pixels in the pancreatic cancer region are 1 and the pixel values of the other regions are 0.

The method for setting the pixel values of the label image of the pancreatic cancer region is not limited to this, and the value that is set for the pixels in the pancreatic cancer region may be 255 or the like. Further, the pixel value of the pancreatic cancer region in the label image of the pancreatic cancer region may be set in accordance with the ratio of the combining ratio determined in step S604. For example, if the combining ratio determined in step S604 is 3:4, the pixel value (label value) of the pancreatic cancer region in the label image of the pancreatic cancer region is set to 4/7. Furthermore, the value may be set such that the pixel values (label values) assigned to the pancreatic cancer region and the other regions smoothly change, based on the distance from the center of the pancreatic cancer region to the edge portion or the edge of the image, for example. In this case, a circumscribed rectangle of the pancreatic cancer region may be set, and the pixel values of the label image may be set to multi-values of Gaussian distribution, of which peak position is the center of the circumscribed rectangle. The pixel values (label values) that are set by this processing may be multiplied by a value based on the combining ratio determined in step S604, and the result may be set as the pixel values.

(Step S609: Execute Learning Processing) In step S609, the learning unit 513 learns the machine learning model to extract the pancreatic cancer region, using the synthetic image generated in step S607 and the teacher label assigned to this image in step S608. The machine learning model here is a deep learning model, such as the convolutional neural network (CNN) and transformers. However, such algorithms as the random forest and the support vector machine (SVM) may be used for the machine learning model.

The data used for the learning of the machine learning model here need not be constituted of only the synthetic image generated in step S607, but the current image and the prior image before combining the images may be used. Particularly in the case where only the pancreatic cancer region is the learning target (that is, in the case where the images indicating healthy cases are not used for learning), the accuracy to detect the pancreatic cancer using the machine learning model increases. On the other hand, if this machine learning model is used, a healthy case may be extracted in error, and the extraction accuracy of a pancreatic cancer may drop. Therefore, it is preferable that the prior image before generating the synthetic image is also used for the learning of the machine learning model.

In Embodiment 2, the pancreatic cancer does not exist in the prior image, so the teacher label assignment unit 512 assigns a teacher label, which indicates the pancreatic cancer does not exist, to the prior image. In the case of using the current image for the learning of the machine learning model, the teacher label assignment unit 512 assigns a teacher label to the current image based on the information on the pancreatic cancer region acquired in step S603. The learning unit 513 may also acquire images other than the current image and the prior image from the database 102 or the storage unit 131, and use these images for the learning of the machine learning model. In this case, the learning unit 513 may acquire teacher labels corresponding to these images from the database 102 or the storage unit 131, or may assign the teacher labels based on the user input via the operation unit 141.

The machine learning model learned by the above processing may be stored in the storage unit 131. For example, if the machine learning model is CNN, such parameters as configuration information of the model, a numeric value of a convolutional kernel, a number of filters, a number of layers, a numeric value of the bias and regulation term, are typically used. The parameters of a machine learning model are not limited to these parameters.

(Step S610: Acquire Inference Image) In step S610, the inference image acquisition unit 514 acquires an inference image, which is used for inference in step S611, from the storage unit 131 or the database 102. For the inference image, the inference image acquisition unit 514 may retrieve an image that satisfies a predetermined condition, from a plurality of images stored in the storage unit 131 or the database 102. The inference image acquisition unit 514 may also acquire the inference image based on the user input via the operation unit 141.

(Step S611: Execute Inference Processing) In step S611, the inference unit 515 acquires the machine learning model learned in step S609, executes the inference processing on the inference image acquired by the inference image acquisition unit 514, and acquires the inference result.

(Step S612: Display Inference Result) In step S612, the output unit 119 displays the inference result acquired in step S611 on the display unit 161 via the display processing unit 151, to present the result to the user. Specifically, along with the inference image, the display processing unit 151 displays the presence/absence of a pancreatic cancer in the inference image, and the position of the pancreatic cancer if present on the display unit 161. The output unit 119 may store the inference result in the storage unit 131 or the database 102. Further, the output unit 119 may determine whether or not the inference result is stored in the storage unit 131 or the database 102, based on the user input via the operation unit 141. An image of the pancreatic cancer region, indicated by the inference result stored in the storage unit 131 or the database 102, may be used as the above mentioned teacher label (label image).

As described above, according to the image processing apparatus 501 of Embodiment 2, learning of the machine learning model, of which extraction accuracy of the early stage pancreatic cancer region has been improved, can be implemented, while alleviating the insufficiency of the teacher data of the early stage pancreatic cancer. Furthermore, in the image processing apparatus 501, the presence/absence and region of a pancreatic cancer can be inferred from the inference image at high accuracy, using the machine learning model acquired by the above processing.

### Modification 2-1

In Embodiment 2, the inference unit 515 executes the pancreatic cancer region extraction (inference) processing in step S611. However, the inference unit 515 may infer whether or not the pancreatic cancer is included in the inference target image. In this case, in step S608, the teacher label assignment unit 512 assigns information indicating whether or not a pancreatic cancer is included in the image, as the teacher label. Then in step S609, the learning unit 513 learns the machine learning model which infers whether or not a pancreatic cancer is included in the image, using the teacher label. For example, in step S611, the inference unit 515 estimates the probability that a pancreatic cancer is included in the inference target image.

In this case, a high value (e.g. 1) is set if a pancreatic cancer is present, a low value (e.g. 0) is set if a pancreatic cancer is absent, and a value adjusted based on the combining ratio determined in step S604 is used as the teacher label. Specifically, if the combining ratio determined in step S604 is 3:4, the value of the teacher label is determined as 4/7. However, the value of the teacher label need not be based on the combining ratio all the time, and values before the adjustment based on the combining ratio (e.g. high value 1, low value 0) may be used as the teacher label.

As described above, according to Modification 2-1, the image processing apparatus 501 can infer the probability of a pancreatic cancer included in the inference target image using the synthetic image generated in step S607. Further, the image processing apparatus 501 can determine whether or not a pancreatic cancer is included in the inference target image by comparing the inferred probability with a predetermined reference value.

### Modification 2-2

In Embodiment 2, the learning unit 513 learns the machine learning model in step S609. However, the inference unit 515 may acquire the machine learning mode from outside of the image processing apparatus 501, and execute the inference for the inference image using the acquired machine learning model. In this case, the image processing apparatus 501 need not include the teacher label assignment unit 512 and the learning unit 513. In Modification 2-2, the machine learning model may be stored in the storage unit 131, and the inference unit 515 may acquire the machine learning model from the storage unit 131.

In Modification 2-2, the image processing apparatus 501 may include a mode to store the machine learning model acquired by executing the processing in steps S601 to S609. Further, the image processing apparatus 501 may include a mode to acquire the stored machine learning model, and execute the processing in steps S610 to S612. Furthermore, the image processing apparatus 501 may switch these modes and execute the processing in the selected mode, based on the user instruction via the operation unit 141, for example.

### Modification 2-3

In Embodiment 2, the teacher label assignment unit 512 assigns a value of the teacher label in accordance with the combining ratio in step S608. However, in the case of assigning the teacher label changing the pixel values (label values) in the pancreatic cancer region in accordance with the combining ratio, an improvement of versatility performance of the machine learning model is expected, but a convergence performance of learning the machine learning model may be diminished. Therefore in the case where the calculation resources (memory amount, learning time) that can be spent for learning of the machine learning model are limited, for example, the teacher label assignment unit 512 may assign the teacher label setting all the pixel values (label values) of the pancreatic cancer label image to 1, regardless the combining ratio. Further, in the case where the combining ratio of the prior image, with respect to the current image is high (e.g. case where the combining ratio of the prior image exceeds 1/2), the teacher label assignment unit 512 may assign the teacher label setting all the pixel values (label values) of the pancreatic cancer label image to 0.

As described above, according to Modification 2-3, the learning processing executed by the learning unit 513 is simplified, whereby a higher efficiency and stability can be expected for the processing executed by the image processing apparatus 501.

Note that the above-described various types of control may be processing that is carried out by one piece of hardware (e.g., processor or circuit), or otherwise. Processing may be shared among a plurality of pieces of hardware (e.g., a plurality of processors, a plurality of circuits, or a combination of one or more processors and one or more circuits), thereby carrying out the control of the entire device.

Also, the above processor is a processor in the broad sense, and includes general-purpose processors and dedicated processors. Examples of general-purpose processors include a central processing unit (CPU), a micro processing unit (MPU), a digital signal processor (DSP), and so forth. Examples of dedicated processors include a graphics processing unit (GPU), an application-specific integrated circuit (ASIC), a programmable logic device (PLD), and so forth. Examples of PLDs include a field-programmable gate array (FPGA), a complex programmable logic device (CPLD), and so forth.

The embodiment described above (including variation examples) is merely an example. Any configurations obtained by suitably modifying or changing some configurations of the embodiment within the scope of the subject matter of the present invention are also included in the present invention. The present invention also includes other configurations obtained by suitably combining various features of the embodiment.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD) TM), a flash memory device, a memory card, and the like.

According to the techniques of the present disclosure, insufficiencies of the patterns of data can be alleviated by generating pseudo images based on the registration information between images.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2024-015884, filed on Feb. 5, 2024, which is hereby incorporated by reference herein in its entirety.

What is claimed is:

1. An image processing apparatus, comprising:
one or more processors; and
a memory storing a program which, when executed by the one or more processors, causes the image processing apparatus to execute:
image acquisition processing to acquire a first image which has an abnormal region in a first region, and a second image which is different from the first image and includes at least the first region, by imaging at least one subject;
region information acquisition processing to acquire region information on the abnormal region in the first image;
registration information acquisition processing to acquire registration information related to registration of the first image and the second image; and
synthetic image generation processing to combine the first image and the second image based on the region information and the registration information, and generate a synthetic image which has a region corresponding to the abnormal region in the first region.

2. The image processing apparatus according to claim 1, wherein
the first region is an organ region,
the second image includes the organ region, and
the organ region in the second image does not have the abnormal region.

3. The image processing apparatus according to claim 1, wherein
the first image and the second image are images acquired by imaging a same subject, and
the second image is an image captured in the past before capturing the first image.

4. The image processing apparatus according to claim 1, wherein
the program, when executed by the one or more processors, further causes the image processing apparatus to execute:
ratio information acquisition processing to acquire combining ratio information related to a combining ratio on the first image and the second image in the synthetic image, and
the synthetic image generation processing generates the synthetic image based on the combining ratio information acquired in the ratio information acquisition processing.

5. The image processing apparatus according to claim 4, wherein
the combining ratio is determined based on imaging information on the first region.

6. The image processing apparatus according to claim 5, wherein
the imaging information includes an abnormality progress degree of the abnormal region, a size of the abnormal region, and at least one of a pixel value of the first region and a pixel value of the abnormal region.

7. The image processing apparatus according to claim 1, wherein
the registration information is information to register the first image and the second image, so that the abnormal region in the first image is included in the first region in the second image.

8. The image processing apparatus according to claim 1, wherein
the program, when executed by the one or more processors, further causes the image processing apparatus to execute:
transformed image generation processing to generate a transformed image by transforming the second image to match with the first image based on the registration information, and
the synthetic image generation processing generates the synthetic image by combining the transformed image and the first image.

9. The image processing apparatus according to claim 1, wherein
the program, when executed by the one or more processors, further causes the image processing apparatus to execute:
transformed image generation processing to generate a transformed image by transforming the first image to match with the second image based on the registration information, and
the synthetic image generation processing generates the synthetic image by combining the transformed image and the second image.

10. The image processing apparatus according to claim 1, wherein
the program, when executed by the one or more processors, further causes the image processing apparatus to execute:
teacher label assignment processing to assign a teacher label that indicate the abnormal region in the synthetic image to the synthetic image generated by the synthetic image generation unit, in order to be used for learning of a machine learning model to infer the abnormal region in an image.

11. The image processing apparatus according to claim 10, wherein
the program, when executed by the one or more processors, further causes the image processing apparatus to execute:
learning processing to learn the machine learning model using the teacher label assigned in the teacher label assignment processing, and the synthetic image.

12. The image processing apparatus according to claim 11, wherein
the program, when executed by the one or more processors, further causes the image processing apparatus to execute:
inference processing to input an inference image to the machine learning model learned in the learning processing and infer the abnormal region in the inference image.

13. The image processing apparatus according to claim 11, wherein
the teacher label indicates, in the synthetic image, a region corresponding to the abnormal region in the first image.

14. The image processing apparatus according to claim 10, wherein
the teacher label is a label value indicating the abnormal region, and
the teacher label assignment processing determines the label value in accordance with a combining ratio of the first image and the second image in the synthetic image.

15. The image processing apparatus according to claim 1, wherein
the program, when executed by the one or more processors, further causes the image processing apparatus to execute:
inference image acquisition processing to acquire an inference image; and
inference processing to input the inference image to a machine learning model learned to infer the abnormal region in an image, using a teacher label which is assigned to the synthetic image and indicates the abnormal region in the synthetic image, and the synthetic image, and execute inference on the abnormal region in the inference image.

16. An image processing method, comprising:
a step of acquiring a first image which has an abnormal region in a first region, and a second image which is different from the first image and includes at least the first region, by imaging at least one subject;
a step of acquiring region information on the abnormal region in the first image;
a step of acquiring registration information related to registration of the first image and the second image; and
a step of combining the first image and the second image based on the region information and the registration information, and generating a synthetic image which has a region corresponding to the abnormal region in the first region.

17. The image processing method according to claim 16, further comprising:
a step of acquiring an inference image; and
a step of inputting the inference image to a machine learning model learned to infer the abnormal region in an image, using a teacher label which is assigned to the synthetic image and indicates the abnormal region in the synthetic image, and the synthetic image, and executing inference on the abnormal region in the inference image.

18. A non-transitory computer readable medium that stores a program, wherein the program causes a computer to execute an image processing method, the image processing method comprising:
a step of acquiring a first image which has an abnormal region in a first region, and a second image which is different from the first image and includes at least the first region, by imaging at least one subject;
a step of acquiring region information on the abnormal region in the first image;
a step of acquiring registration information related to registration of the first image and the second image; and
a step of combining the first image and the second image based on the region information and the registration information, and generating a synthetic image which has a region corresponding to the abnormal region in the first region.
